(19) United States
(12) Patent Application Publication
(10) Pub. No.: US 2026/0256984 A1
(43) Pub. Date: Sep. 3, 2026

(54) Structural Composite Engineered Artificial Bone and Fabrication Method Thereof

(71) Applicant: Shenzhen YuanShengLin Technology Company Limited, Shenzhen (CN)

(72) Inventors: Tenghui Zeng, Shenzhen (CN); Baoming Qiu, Shenzhen (CN); Yupeng Lan, Shenzhen (CN)

(21) Appl. No.: 19/298,419

(22) Filed: Aug. 13, 2025

(30) Foreign Application Priority Data
Feb. 28, 2025 (CN) 202510247196.2

(51) Int. Cl. A61L 27/28

(52) U.S. Cl. CPC A61L 27/28; A61L 2430/02

(57) ABSTRACT

Disclosed is a structural composite engineered artificial bone based on architectural engineering and fabrication method thereof. The structural composite engineered artificial bone includes a bioactive material and a mechanical support scaffold. The bioactive material serves as a matrix, with the mechanical support scaffold incorporated therein. The two components are closely combined with each other to form an integrated artificial bone tissue composite. The mechanical support scaffold is configured to provide mechanical support for the integrated artificial bone tissue composite to meet load-bearing requirements during bone repair. The artificial bone tissue composite can also be further modified through surface treatment and/or by a carrying structure with an osteogenic material sustained-release system and/or drug sustained-release system arranged inside, to achieve one or more functions of meeting clinical needs for anti-inflammation, anti-infection, anti-tumor, anti-osteoporosis, and osseointegration.

### CROSS-REFERENCE TO RELATED APPLICATIONS

The present application claims priority of Chinese Patent Application No. 202510247196.2, filed on Feb. 28, 2025, the entire contents of which are hereby incorporated by reference.

### TECHNICAL FIELD

The disclosure belongs to the field of biomedical engineering, and in particular relates to a structural composite engineered artificial bone and fabrication method thereof.

### BACKGROUND

With the aging of the population and the increasing prevalence of orthopedic diseases, the treatment of bone defects has received increasing attention, particularly in the spine and load-bearing sites of the extremities. Conventional approaches typically involve the use of bone repair materials or support structures for integrated restoration. However, both methods present certain limitations as follows.

Regarding bone repair materials—currently available materials with relatively strong osseointegration capacity primarily include calcium matrix material, silicon matrix materials, polymers, and magnesium metal. While these materials exhibit certain osseointegration capabilities, their mechanical properties remain unsatisfactory and fail to meet the mechanical demands of weight-bearing segments. For example, calcium matrix materials have low tensile strength and bending strength, are prone to brittle fracture, and are difficult to withstand complex loads. Calcium matrix materials are also prone to fatigue failure, particularly in high-intensity load-bearing applications. Silicon matrix materials have weak mechanical properties, typically with low compressive strength and toughness, making them inadequate for sustaining prolonged mechanical loading in high-intensity load-bearing applications. They are also highly brittle and thus prone to crack expansion and fracture under impact or fatigue loads. Magnesium metal exhibits certain osteoinductive properties, but its uncontrolled degradation and degradation-related toxicity limit its standalone application. Currently, it can only be utilized for surface modification of materials or through sustained-release systems.

Furthermore, the composite materials combining polymer with calcium or silicon matrix face two main challenges: First, there is a conflict between mechanical and biological properties. The composite materials are required to simultaneously possess both high mechanical strength and excellent bioactivity, yet achieving an optimal balance between these two characteristics remains technically difficult. Second, the interfacial bonding strength between the polymer and the calcium or silicon matrix is often insufficient, which may lead to delamination, peeling, or failure of the composite material. While current composite osseointegration materials primarily focus on improving osseointegration capabilities along with additional functions such as anti-inflammatory, anti-infection, and anti-tumor properties, they still fail to adequately meet the bone replacement and repair requirements of load-bearing segments.

For load-bearing structures, microporous titanium alloy materials, which have good mechanical properties and biocompatibility, are the most widely used. However, they also have several following drawbacks. First, the bonding strength between titanium alloy and bone is weak, resulting in insufficient osseointegration capability and frequent prosthetic loosening. Second, pure titanium alloy has a high elastic modulus and is prone to bone collapse and sinking of the titanium alloy at the interface. Third, although the microporous structure demonstrates certain osseointegration capability, this capability is confined to a range of 1-3 millimeters at the interface. It fails to achieve deep osseointegration, thus resulting in poor interfacial mechanical properties. Moreover, the above-mentioned materials do not possess anti-infection and anti-inflammatory capabilities. Composite materials combined with titanium alloy are currently limited to surface modification.

In summary, there remains currently a lack of artificial bone substitutes that can achieve good osseointegration, meet the mechanical load-bearing requirements of load-bearing segments, and possess a variety of bioactive functions. These limitations prevent them from satisfying the diverse clinical needs. Therefore, there is an urgent need for a new type of structural composite engineered artificial bone material that incorporates principles of architectural engineering.

### SUMMARY

The objective of the present disclosure is to provide a structural composite engineered artificial bone and a preparation method thereof, which takes into account both the osseointegration capacity and the mechanical properties of the material.

The present disclosure is implemented as follows: a structural composite engineered artificial bone including a bioactive material and a mechanical support scaffold; wherein the bioactive material serves as a matrix, with the mechanical support scaffold incorporated therein. The two components are closely combined with each other to form an integrated artificial bone tissue composite. The mechanical support scaffold is configured to provide mechanical support for the integrated artificial bone tissue composite so as to meet the load-bearing requirements during bone repair.

The present disclosure further provides a fabrication method of the aforementioned structural composite engineered artificial bone, including the following steps:
preparing a mechanical support scaffold;
performing surface treatment on the mechanical support scaffold;
combining the mechanical support scaffold with a bioactive material by means of high-temperature sintering, 3D printing, plasma spraying, or filling and curing to form an integrated artificial bone tissue composite;
constructing a carrying structure on the surface and/or interior of the integrated artificial bone tissue composite; and
embedding a sustained-release system containing osteogenic materials and/or drugs with corresponding functions into the carrying structure.

Compared with the prior art, the present disclosure has the following beneficial effects:
1. The structural composite engineered artificial bone provided by the present disclosure incorporates a built-in mechanical support scaffold, which provides sufficient mechanical support and toughness to meet the load-bearing requirements during bone repair. This provides solutions for extensive bone defects, structural filling and support of bone, as well as treatment of long-term load-bearing sites.
2. The mechanical support scaffold is closely incorporated within the bioactive material, which enhances the integration with bone tissue. As a result, the osseointegration effect is optimized through the natural biological reactions with the bone tissue.

### BRIEF DESCRIPTION OF DRAWINGS

FIG. 1 is a perspective schematic diagram of a structural composite engineered artificial bone according to an embodiment of the present disclosure;

FIG. 2 is a schematic top view of a structural composite engineered artificial bone according to an embodiment of the present disclosure;

FIG. 3 is a schematic side view of a structural composite engineered artificial bone according to an embodiment of the present disclosure;

FIG. 4 is a schematic cross-sectional view of a structural composite engineered artificial bone according to an embodiment of the present disclosure;

FIG. 5A is a schematic diagram of a shape of a mechanical support scaffold according to an embodiment of the present disclosure;

FIG. 5B is a schematic diagram of a shape of a mechanical support scaffold according to an embodiment of the present disclosure;

FIG. 5C is a schematic diagram of a shape of a mechanical support scaffold according to an embodiment of the present disclosure;

FIG. 5D is a schematic diagram of a shape of a mechanical support scaffold according to an embodiment of the present disclosure;

FIG. 5E is a schematic diagram of a shape of a mechanical support scaffold according to an embodiment of the present disclosure;

FIG. 5F is a schematic diagram of a shape of a mechanical support scaffold according to an embodiment of the present disclosure;

FIG. 5G is a schematic diagram of a shape of a mechanical support scaffold according to an embodiment of the present disclosure;

FIG. 5H is a schematic diagram of a shape of a mechanical support scaffold according to an embodiment of the present disclosure;

FIG. 6 is a cross-sectional schematic diagram of a grid-shaped mechanical support scaffold according to an embodiment of the present disclosure;

FIG. 7A is a schematic diagram illustrating an application of a structural composite engineered artificial bone according to an embodiment of the present disclosure between two vertebrae;

FIG. 7B is a schematic diagram illustrating an application of a structural composite engineered artificial bone according to an embodiment of the present disclosure in a lower limb bone defect.

Reference signs: 1, bioactive material; 2, mechanical support scaffold; 21, inner passage; 3, artificial bone tissue composite; 31, carrying structure; 32, orifice; 33, micropore; 4, drug sustained-release sphere.

### DESCRIPTION OF EMBODIMENTS

In order to make the objectives, technical solutions and advantages of the present disclosure clearer, the present disclosure will be further described in detail below in reference to drawings and embodiments. It should be understood that the specific embodiments described herein are only used to explain the present disclosure rather than to limit the present disclosure.

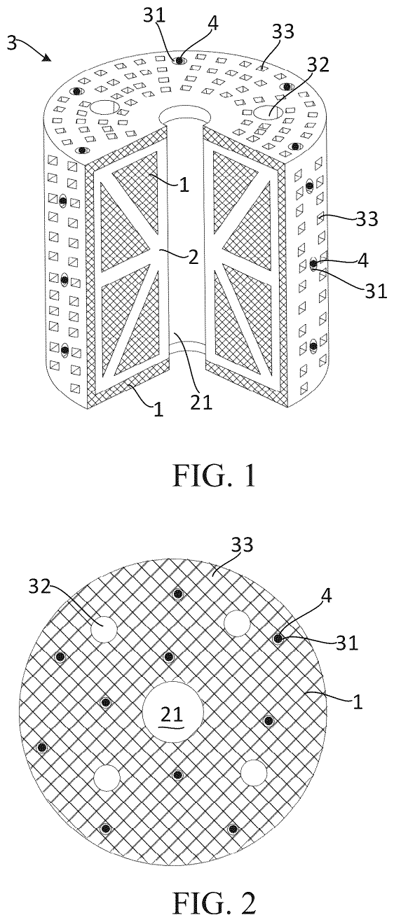

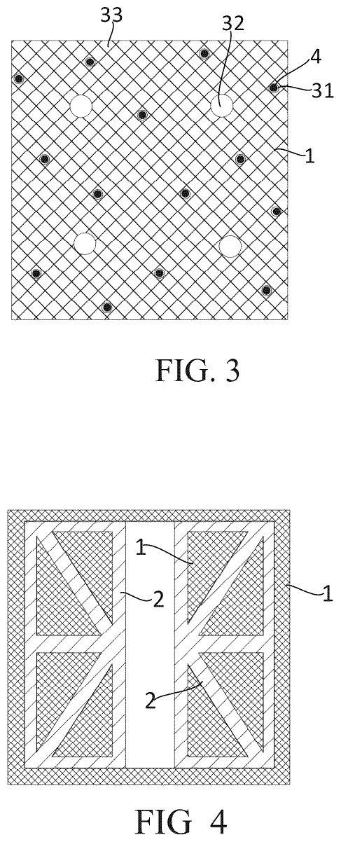

The present embodiment provides a structural composite engineered artificial bone with a structure illustrated in FIGS. 1 to 4. The structural composite engineered artificial bone includes a bioactive material 1 and a mechanical support scaffold 2, wherein the bioactive material 1 serves as the matrix, with the mechanical support scaffold 2 incorporated therein. The two components are closely combined with each other to form an integrated artificial bone tissue composite 3. Specifically, the bioactive material 1 may be filled within the mechanical support scaffold 2, or the bioactive material: (1) may fill the interior of the mechanical support scaffold; and (2) subsequently cover the surface thereof. The bioactive material 1 provides good osseointegration capability, and the osseointegration effect is optimized through the natural biological reactions with the bone tissue. Meanwhile, the mechanical support scaffold 2 provides mechanical support and toughness for the integrated artificial bone tissue composite 3 to meet the load-bearing requirements during bone repair. This provides solutions for extensive bone defects, structural filling and support of bone, as well as treatment of long-term load-bearing sites.

The close combination between the bioactive material 1 and the mechanical support scaffold 2 is analogous to the “reinforced concrete” structure in construction engineering. Specifically, this can be achieved through high-temperature sintering, 3D printing, plasma spraying or filling and curing.

Specifically, the bioactive material 1 may be bioceramic composite particles or porous bioglass, wherein the bioceramic composite particles may be made of one or more of following materials: calcium hydroxide, calcium phosphate, or nano-silica. The mechanical support scaffold 2 is made of, but not limited to, one or more of the following materials: tantalum alloy, magnesium alloy, polylactic acid (PLA) and its copolymers, ceramics and their composites, polymers, carbon fibers and their composites.

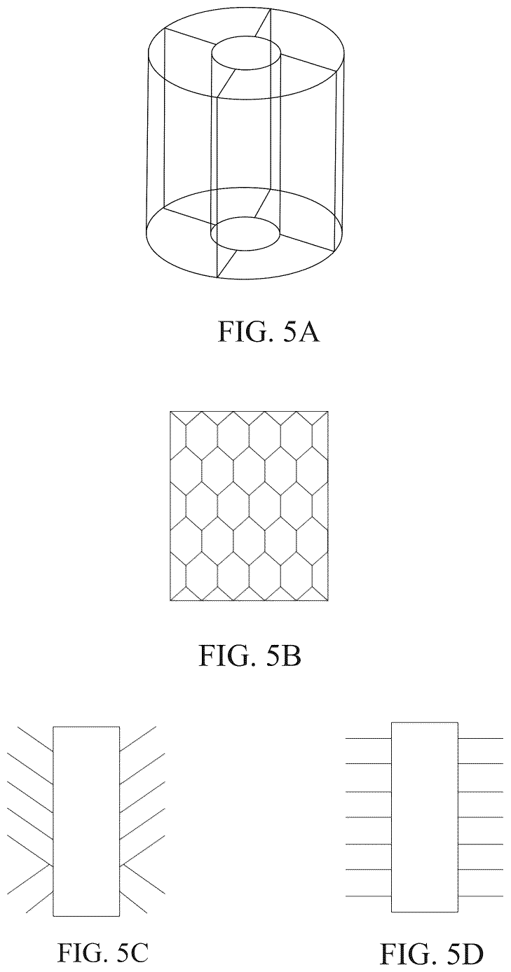

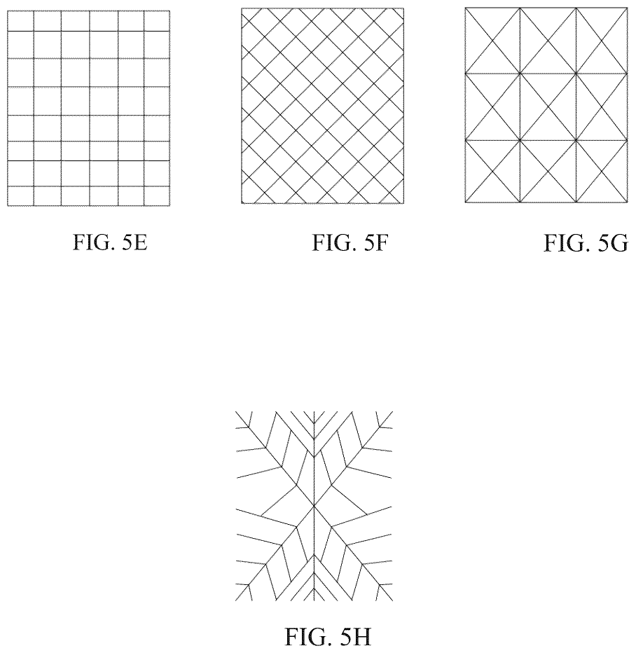

The specific shape of the mechanical support scaffold 2 is not restricted and may be designed through mechanical calculation to be any shape or style that can provide sufficient mechanical support for the entire composite artificial bone. For example, it may be a fence-like shape as shown in FIG. 5A, a honeycomb-like shape as shown in FIG. 5B, a bifurcated shape as shown in FIGS. 5C and 5D, a grid-like shape as shown in FIGS. 5E, 5F, and 5G, a dendritic-like shape as shown in FIG. 5H, and so on.

Further, an osteogenic material sustained-release system and/or a drug sustained-release system may be loaded at the surface and/or interior of the integrated artificial bone tissue composite 3. As an implementation, a carrying structure 31 may be designed on the surface and/or interior of the integrated artificial bone tissue composite 3 to accommodate the osteogenic material sustained-release system and/or the drug sustained-release system. As another implementation, the osteogenic material sustained-release system and/or the drug sustained-release system may also be coated on the surface of the carrying structure 31. The osteogenic material and/or drug in the sustained-release system is continuously and slowly released, and the drug therein may be selected according to the specific condition of the patient, so as to provide corresponding functions in different application scenarios.

Preferably, the osteogenic material is selected from the following: a porous calcium phosphate matrix material, a silicon matrix biomaterial, a polymer, a collagen and protein matrix material, chitosan, a carbon matrix material, a nanocomposite polymer composite material, wherein the silicon matrix biomaterial may include bioglass.

Further, the release rate of the osteogenic material and/or drug within the sustained-release system is designed to be gradient in the spatial dimension, wherein the release rate of the osteogenic material and/or drug near a bone contact end is greater than the release rate of the osteogenic material and/or drug far from the bone contact end. This gradient release design helps maintain the localized drug concentration, improve the drug utilization efficiency, and enable personalized treatment.

In terms of structure, the aforementioned osteogenic material sustained-release system and/or drug sustained-release system includes, but is not limited to, at least one of the following: a coating on the outer surface of the integrated artificial bone tissue composite 3, and microspheres or microcapsules embedded within the carrying structure 31.

In the present application, the release rate of the above osteogenic material sustained-release system and/or drug sustained-release system, in addition to having a gradient design in the spatial dimension as described above, may further have a gradient design in the temporal dimension as well, as exemplified in the following examples with respect to the microsphere, coating, and microcapsule sustained-release systems, respectively.

For the case where the osteogenic material release system and/or the drug release system are microspheres, as shown in FIGS. 1 to 3, the aforementioned microspheres may be drug sustained-release spheres 4, which provide sustained release of osteogenic material and/or drug, thereby accelerating bone healing and repair. Each drug sustained-release sphere 4 has a core and a coating layer covering the core. The coating layer is made of human-absorbable materials, which may include one or more of the following: ceramic materials, glass materials, biodegradable polymers, natural polymer materials, chitosan, nanomaterials, hydrogel materials, and ceramic-polymer composite materials. The core contains one or more of the following: osteogenic materials, drugs, magnesium alloys, zinc alloys, hydrogels, and polymers. For embodiments where the polymer is used as the coating layer and the core, the drug is encapsulated between the coating layer and the core, as well as inside the core. The composition ratio of the polymer is adjusted according to the desired release time. The coating layer degrades faster, so that the drug between the coating layer and the core is released first. The core degrades more slowly, so that the drug inside the core is released later, thus achieving the release of the drug in a time gradient.

For embodiments where the osteogenic material sustained-release system and/or the drug sustained-release system described above is a coating, the coating may have a multilayer structure. The multilayered coating is constructed on the surface of the composite artificial bone by layer-by-layer coating or 3D printing, with each layer of material containing the drug. Each layer of material exhibits distinct degradation rates, resulting in different gradient release.

For embodiments where the osteogenic material sustained-release system and/or drug release system described above is a microcapsule sustained-release system, drug-carrying nanoparticle microcapsules may be utilized to achieve phased drug release under external stimuli—including magnetic field, pH, enzyme, or temperature—thereby achieving gradient release. For example, in phase 1 treatment, the artificial bone site of the patient is subjected to conditions of a first pH value, a first type of enzyme, and a first temperature, and in phase 2 treatment, the artificial bone site of the patient is subjected to conditions of a second pH value, a second type of enzyme, and a second temperature.

As described above, the carrying structure 31 is disposed on the surface, interior, or both the surface and interior of the integrated artificial bone tissue composite 3. As an example, FIGS. 1 to 3 illustrate that the carrying structure 31 may be a cavity structure with at least one port communicating with the outer surface of the integrated artificial bone tissue composite 3. This design facilitates the placement of a sustained-release system from the exterior of the integrated artificial bone tissue composite 3, and also enables the effusion of osteogenic materials and/or drugs from the sustained-release system to contact bone tissue.

Furthermore, it is also possible to introduce micropores 33 in the bioactive material 1. Such micropores 33 may be structured for bone tissue to grow into, and the pore size and porosity of the micropores 33 meet the requirements of biomechanics and bone tissue regeneration. Preferably, the porosity of the micropores 33 is in the bioactive material 1 is in a range of 50% to 80%, with a pore size ranging from 100 μm to 500 μm. It should be noted that the specific shape of the micropore 33 is not limited to a square pore as shown in FIG. 1, or a pore of other shapes, such as a circle. Alternatively, the entire bioactive material 1 may be designed in a sponge-like configuration, and the pores in the sponge serve as the micropores 33. For example, the black dots in the bioactive material 1 as shown in FIGS. 1 and 4 are micropores 33.

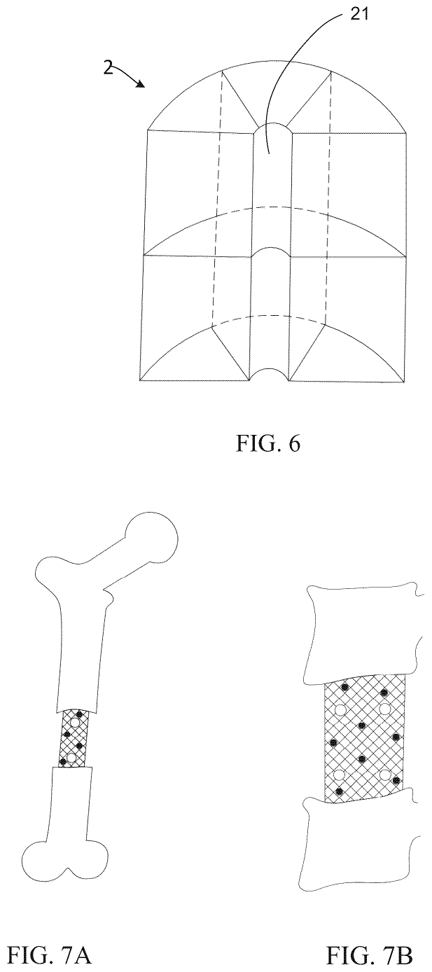

FIG. 6 shows a cross-sectional structure of a grid-like mechanical support scaffold, wherein the middle portion of the mechanical support scaffold 2 is hollow to form an inner passage 21, and the inner passage 21 penetrates through the top and bottom of the mechanical support scaffold 1. The design of this inner passage 21 serves to accelerate drug delivery along the longitudinal direction of the artificial bone while facilitating the ingrowth of bone tissue and blood vessels. The internal supporting structure of the mechanical support scaffold 2 is formed by interconnection of plates, or by interconnection of plates and rods. It should be noted that a carrying structure 31 may be provided on the plate of the mechanical support scaffold 2, and the bioactive material 1 may be applied to cover the surface of the plate of the mechanical support scaffold 2.

Please refer to FIGS. 1 and 4, which show the cross-sectional structure of the artificial bone tissue composite formed by filling a bioactive material within a mechanical support scaffold according to this embodiment. The integrated artificial bone tissue composite 3 has at least two structural layers. Furthermore, the interior of the integrated artificial bone tissue composite 3 is provided with multiple orifices 32 for blood vessel ingrowth. The orifices 32 are aligned with the inner hole 21 in direction and both penetrate the top and bottom of the mechanical support scaffold 1. The orifices 32 are relatively narrower than the inner passage 21. The orifices 32 and the inner passage 21 are communicated through micropores 33 and extend to the outer surface of the artificial bone tissue composite 3.

This embodiment further provides a method for preparing a structural composite engineered artificial bone, including the following steps:
preparing the mechanical support scaffold 2 by specialized mold forming technology or 3D printing technology;
performing surface treatment on the mechanical support scaffold 2 to create an uneven surface morphology; which facilitates the formation of a close combination with the bioactive material 1. This may be achieved through methods such as spraying, laser etching, chemical etching, and coating treatment.
combining the mechanical support scaffold 2 with the bioactive material 1 to form an integrated artificial bone tissue composite 3 by means of high-temperature sintering, 3D printing, plasma spraying, or filling and curing;
constructing a porous structure 31 on the surface and/or interior of the integrated artificial bone tissue composite 3 during the manufacturing process of or following the formation of the artificial bone tissue composite 3; and
embedding a sustained-release system containing osteogenic materials and/or drugs with different functions into the porous structure 31.

In summary, the composite artificial bone of this embodiment has at least the following advantages compared with traditional bone repair materials:
1. The mechanical support scaffold 2 provides sufficient mechanical support to meet the load-bearing requirements during bone repair.
2. The integration of the mechanical support scaffold 2 with the bioactive material 1 enhances osseointegration while ensuring controlled degradation. Meanwhile, these bioactive materials degrade at a rate synchronized with bone healing progression, thereby preventing long-term artificial material retention. This osseointegration effect is optimized through the natural biological reactions with the bone tissue.
3. The incorporated carrying structure carries a sustained-release system, which contains osteogenic materials and/or drugs. The continuous and slow release of these osteogenic materials and/or drugs from the sustained-release system accelerates bone regeneration and repair processes.
4. As shown in FIGS. 7A and 7B, the composite artificial bone provides new solutions for different application scenarios, such as extensive bone defects, structural filling and support of bone, as well as treatment of long-term load-bearing sites.

The above description is only an optional embodiment of the present application and is not intended to limit the present application. For those skilled in the art, the present application can have various modifications and changes. Any modification, equivalent replacement, and improvement made within the spirit and principle of the present application shall be included within the protection scope of the present application.

What is claimed is:

1. A structural composite engineered artificial bone, comprising
a bioactive material serving as a matrix; and
a mechanical support scaffold incorporated within the bioactive material;
wherein the bioactive material and the mechanical support scaffold are integrally combined with each other to form an integrated artificial bone tissue composite, and the mechanical support scaffold is configured to provide mechanical support for the integrated artificial bone tissue composite to meet load-bearing requirements during bone repair.

2. The structural composite engineered artificial bone according to claim 1, wherein the mechanical support scaffold is in a form of a honeycomb structure, a bifurcated structure, a grid structure, a fence structure, or a dendritic structure, and the mechanical scaffold is a biodegradable composite scaffold or a non-degradable scaffold.

3. The structural composite engineered artificial bone according to claim 1, wherein an osteogenic material sustained-release system and/or a drug sustained-release system is loaded on a surface and/or an interior of the integrated artificial bone tissue composite.

4. The structural composite engineered artificial bone according to claim 3, wherein the surface and/or interior of the integrated artificial bone tissue composite is provided with a carrying structure, the carrying structure is configured to accommodate the osteogenic material sustained-release system and/or drug sustained-release system; or
the osteogenic material sustained-release system and/or drug sustained-release system is coated on a surface of the carrying structure.

5. The structural composite engineered artificial bone according to claim 4, wherein the carrying structure is a cavity structure, the cavity structure is located on the surface of the integrated artificial bone tissue composite; or
the cavity structure extends from the surface to the interior of the integrated artificial bone tissue composite.

6. The structural composite engineered artificial bone according to claim 3, wherein a release rate of the sustained-release system is designed to be gradient in a spatial dimension, wherein the release rate of the osteogenic material and/or drug near a bone contact end is greater than the release rate of the osteogenic material and/or drug far from the bone contact end.

7. The structural composite engineered artificial bone according to claim 6, wherein the osteogenic material sustained-release system and/or drug sustained-release system comprises at least one of the following: a coating on an outer surface of the integrated artificial bone tissue composite, and microspheres and microcapsules arranged within the carrying structure.

8. The structural composite engineered artificial bone according to claim 1, wherein the bioactive material has micropores for bone tissue ingrowth, a porosity of the micropores on the bioactive material is in a range of 50% to 80%, with a pore size ranging from 100 μm to 500 μm.

9. The structural composite engineered artificial bone according to claim 1, wherein the interior of the integrated artificial bone tissue composite is provided with orifices for blood vessel ingrowth and to provide growth space for bone tissue, and the orifices extend to the outer surface of the integrated artificial bone tissue composite.

10. A fabrication method of the structural composite engineered artificial bone according to claim 1, comprising the following steps:
preparing a mechanical support scaffold;
performing surface treatment on the mechanical support scaffold;
combining the mechanical support scaffold with a bioactive material to form an integrated artificial bone tissue composite by means of high-temperature sintering, 3D printing, plasma spraying, or filling and curing;
constructing a carrying structure on a surface and/or interior of the integrated artificial bone tissue composite; and
embedding a sustained-release system containing osteogenic materials and/or drugs with corresponding functions into the carrying structure.

11. The structural composite engineered artificial bone according to claim 4, wherein a release rate of the sustained-release system is designed to be gradient in a spatial dimension, wherein the release rate of the osteogenic material and/or drug near a bone contact end is greater than the release rate of the osteogenic material and/or drug far from the bone contact end.

12. The structural composite engineered artificial bone according to claim 5, wherein a release rate of the sustained-release system is designed to be gradient in a spatial dimension, wherein the release rate of the osteogenic material and/or drug near a bone contact end is greater than the release rate of the osteogenic material and/or drug far from the bone contact end.

13. A fabrication method of the structural composite engineered artificial bone according to claim 2, comprising the following steps:
preparing a mechanical support scaffold;
performing surface treatment on the mechanical support scaffold;
combining the mechanical support scaffold with a bioactive material to form an integrated artificial bone tissue composite by means of high-temperature sintering, 3D printing, plasma spraying, or filling and curing;
constructing a carrying structure on a surface and/or interior of the integrated artificial bone tissue composite; and
embedding a sustained-release system containing osteogenic materials and/or drugs with corresponding functions into the carrying structure.

14. A fabrication method of the structural composite engineered artificial bone according to claim 3, comprising the following steps:
preparing a mechanical support scaffold;
performing surface treatment on the mechanical support scaffold;
combining the mechanical support scaffold with a bioactive material to form an integrated artificial bone tissue composite by means of high-temperature sintering, 3D printing, plasma spraying, or filling and curing;
constructing a carrying structure on a surface and/or interior of the integrated artificial bone tissue composite; and
embedding a sustained-release system containing osteogenic materials and/or drugs with corresponding functions into the carrying structure.

15. A fabrication method of the structural composite engineered artificial bone according to claim 4, comprising the following steps:
preparing a mechanical support scaffold;
performing surface treatment on the mechanical support scaffold;
combining the mechanical support scaffold with a bioactive material to form an integrated artificial bone tissue composite by means of high-temperature sintering, 3D printing, plasma spraying, or filling and curing;
constructing a carrying structure on a surface and/or interior of the integrated artificial bone tissue composite; and
embedding a sustained-release system containing osteogenic materials and/or drugs with corresponding functions into the carrying structure.

16. A fabrication method of the structural composite engineered artificial bone according to claim 5, comprising the following steps:
preparing a mechanical support scaffold;
performing surface treatment on the mechanical support scaffold;
combining the mechanical support scaffold with a bioactive material to form an integrated artificial bone tissue composite by means of high-temperature sintering, 3D printing, plasma spraying, or filling and curing;
constructing a carrying structure on a surface and/or interior of the integrated artificial bone tissue composite; and
embedding a sustained-release system containing osteogenic materials and/or drugs with corresponding functions into the carrying structure.

17. A fabrication method of the structural composite engineered artificial bone according to claim 6, comprising the following steps:
preparing a mechanical support scaffold;
performing surface treatment on the mechanical support scaffold;
combining the mechanical support scaffold with a bioactive material to form an integrated artificial bone tissue composite by means of high-temperature sintering, 3D printing, plasma spraying, or filling and curing;
constructing a carrying structure on a surface and/or interior of the integrated artificial bone tissue composite; and
embedding a sustained-release system containing osteogenic materials and/or drugs with corresponding functions into the carrying structure.

18. A fabrication method of the structural composite engineered artificial bone according to claim 7, comprising the following steps:
preparing a mechanical support scaffold;
performing surface treatment on the mechanical support scaffold;
combining the mechanical support scaffold with a bioactive material to form an integrated artificial bone tissue composite by means of high-temperature sintering, 3D printing, plasma spraying, or filling and curing;
constructing a carrying structure on a surface and/or interior of the integrated artificial bone tissue composite; and
embedding a sustained-release system containing osteogenic materials and/or drugs with corresponding functions into the carrying structure.

19. A fabrication method of the structural composite engineered artificial bone according to claim 8, comprising the following steps:
preparing a mechanical support scaffold;
performing surface treatment on the mechanical support scaffold;
combining the mechanical support scaffold with a bioactive material to form an integrated artificial bone tissue composite by means of high-temperature sintering, 3D printing, plasma spraying, or filling and curing;
constructing a carrying structure on a surface and/or interior of the integrated artificial bone tissue composite; and
embedding a sustained-release system containing osteogenic materials and/or drugs with corresponding functions into the carrying structure.

20. A fabrication method of the structural composite engineered artificial bone according to claim 9, comprising the following steps:
preparing a mechanical support scaffold;
performing surface treatment on the mechanical support scaffold;
combining the mechanical support scaffold with a bioactive material to form an integrated artificial bone tissue composite by means of high-temperature sintering, 3D printing, plasma spraying, or filling and curing;
constructing a carrying structure on a surface and/or interior of the integrated artificial bone tissue composite; and
embedding a sustained-release system containing osteogenic materials and/or drugs with corresponding functions into the carrying structure.
